(19) United States
(12) Patent Application Publication
(10) Pub. No.: US 2004/0265949 A1
(43) Pub. Date: Dec. 30, 2004

(54) Method and building blocks for preparing C-terminally labelled peptides

(76) Inventors: Peter David White, Southwell (GB); Jorg Karl Wilheim Beythien, Budendorf (CH)

Correspondence Address: FRELING E. BAKER, BROWN MARTIN HALLER & MCCLAIM, 1660 UNION STREET, SAN DIEGO, CA, 92101, US

(21) Appl. No.: 10/607,175

(22) Filed: Jun. 26, 2003

(51) Int. Cl. C12P 21/06; C07K 1/02

(52) U.S. Cl. 435/68.1; 530/333

(57) ABSTRACT

A method for preparing C-terminally labelled peptides and building blocks to be used in this synthesis includes a trivalent nitrogen atom having at least one device for attachment to a solid support, one device for the attachment of amino acids and one device for attachment of a label, whereby the device for the attachment of amino acids and/or the device for the attachment of a label is a linker, e.g. an alkyl- or polyethyleneglycol- linker.

The present invention relates to a method for preparing C-terminally labelled peptides and building blocks to be used in this synthesis. The building blocks according to the present invention ensure an easy synthesis of C-terminally labelled peptides. They comprise a trivalent nitrogen atom having at least one means for the attachment to a solid support, one means for the attachment of amino acids and one means for the attachment of a label, whereby the means for the attachment of amino acids and/or the means for the attachment of a label comprise a linker, e.g. an alkyl- or polyethyleneglycol- linker.

### BACKGROUND

It is believed that approximately 2% of the human genome encodes for proteases [A. J. Barrett (1998) Handbook of Proteolytic enzymes, Academic Press, London]. Proteases are essential to all aspects of life and are important targets for drug development. Indeed, protease inhibitors are used to treat diseases ranging from AIDS to hypertension. Now that the sequence of the human genome is known, the race is on to define the biological activity of the many new proteases revealed. With this has come the need for efficient methods for determining protein specificity and activity.

Fluorescence-quenched peptide substrates are extremely useful tools for this purpose, particularly for determining the activity of endoproteases where conventional carboxy-terminally labelled substrates are not always appropriate [M. Taliani, et al. (1997) Lett. Pept. Sci., 4, 101 and references therein]. Such substrates typically contain a fluorophore and a quencher group attached to either side of the cleavage site. In the intact molecule, the natural fluorescence of the fluorophore is suppressed by the proximity of the quench through a process called fluorescence resonance energy transfer (FRET). Upon cleavage of the substrate by a protease, the quench and fluorophore become separated, leading to an increase in fluorescence, which can then be detected spectrophotometrically. Sensitivity is determined primarily by the distance between fluorophore and quench, which should be in the range 10-100 Å, and the extent of overlap between the absorbance spectrum of the quench and the emission spectrum of the fluorophore.

The standard methods for preparing such peptides consist of:

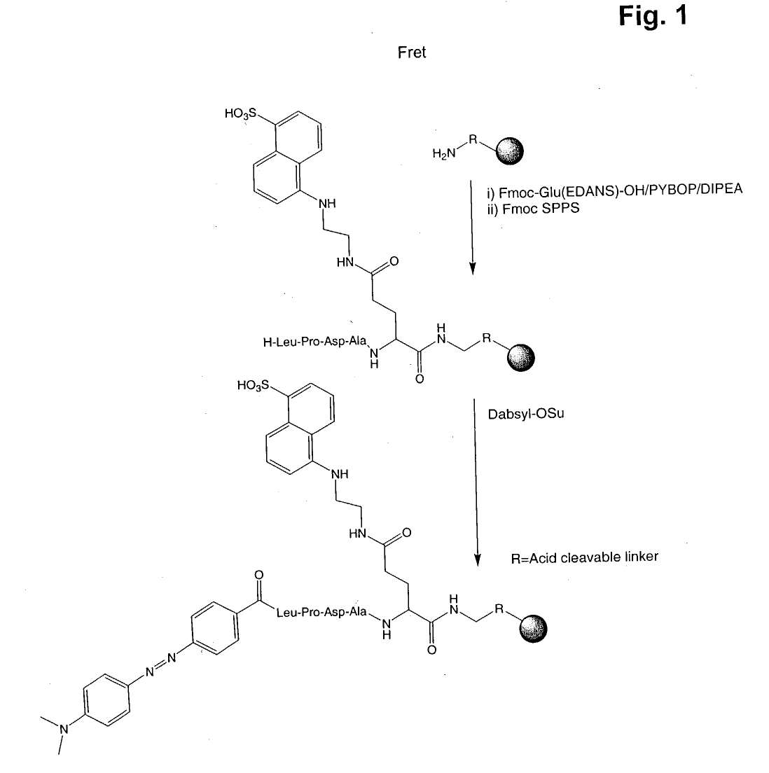

1) incorporating an amino acid into the peptide chain during solid phase synthesis bearing a fluorophore or quencher group in the side chain, and then adding the complementary chromogenic moiety to the N-terminal residue prior to cleavage [L. L. Maggiora, et al. (1992) J. Med. Chem., 35, 3727], as exemplified in FIG. 1;

2) using a protecting group which can be selectively removed from the side-chain of a particular residue and then introducing the chromogenic moiety [M. Taliani, et al. (1997) Lett. Pept. Sci., 4, 101].

These methods have one or more of the following drawbacks:

1) the process introduces an unnatural amino acid into the peptide, which may effect the conformation and binding properties of the peptide substrate;

2) requires extra synthetic steps;

3) introduction of bulky labelled amino acids can be difficult, particularly if the peptide is already hindered.

4) once the label is incorporated it cannot be exchanged for a more/less sensitive label at a later time.

What is therefore required is a flexible and versatile method which allows any fluorescence-quenched or generally C-terminally labelled peptide substrate to be prepared by solid phase synthesis in single substrate, substrate array and library format with any fluorophore/quench pairing, which, ideally, can be varied after the synthesis is completed.

### SUMMARY OF THE INVENTION

It has been found that a building block comprising a trivalent nitrogen atom and at least one linker can be favourably used for the preparation of C-terminally labelled peptides via solid phase synthesis. The building block according to the present invention ensures a minimum of synthetic steps and high flexibility concerning the type of the label and the time at which the label is introduced into the peptide.

The invention therefore relates to a building block for preparing C-terminally labelled peptides by solid phase peptide synthesis according to formula I

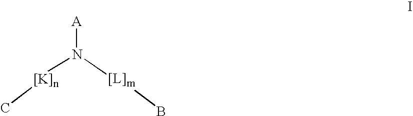

wherein

A is a functionality for the attachment to a solid support or a functionality already comprising a solid support

B is a functionality for the attachment of one or more amino acids or peptides or a functionality already comprising one or more amino acids or peptides

C is a functionality for the attachment of one or more labels or a functionality already comprising one or more labels,

K and L are independently from one another a linear or branched, substituted or unsubstituted alkyl chain with at least two C-atoms, whereby one or more non-neighbouring C-atoms might be substituted by O, NH, N-Alkyl, N-Aryl, S, a carbonyl group, an amide group, ester group and the like and/or neighbouring C-atoms might also be connected via a double or triple bond.

m, n are 0 or 1, whereby m+n is at least 1.

In a preferred embodiment, B is an amino protecting group or a protected amino group, i.e. an amino group carrying a protecting group.

In a preferred embodiment, C comprises one or more labels.

In a preferred embodiment, m+n is 1, that means that only one of the functionalities B and C is connected to the central trivalent nitrogen atom via a linker K or L respectively. The other one is directly bound to the central trivalent nitrogen atom.

In another preferred embodiment, K and L are independently from one another are C₂—C₈-alkyl or —(O—CH₂—CH₂—)_{q} with q=1 to 20.

In another preferred embodiment, A is a residue according to formula II

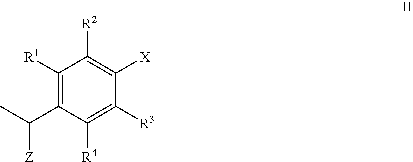

whereby

R¹, R², R³ and R⁴ independently from one another are H, C₁—C₈ alkyl, C₁—C₈ alkoxy, C₅—C₁₈ aryl or heteroaryl, C₅—C₁₈ aryloxy or heteroaryloxy, NO₂, F, Cl, Br or 1, preferably C₁—C₄-alkyl or C₁—C₄ alkyloxy

X is a functionality for attachment to the solid support or a functionality already comprising a solid support, preferably a residue according to formula III

—D —R⁵—E III

with

D being CH₂, S, NH or O

R⁵ being C1—C10 alkyl

E being COOH, OH, SH, NCS, NCO, NH₂, halide (Br,Cl,I) or the solid support

Z is H, C₁—C₈-alkyl, C₅—C₂₀ aryl or C₅—C₂₀ heteroaryl.

The present invention further relates to a method for preparing C-terminally labelled peptides by solid phase synthesis using a building block according to the present invention which does not comprise a label by

a) optionally loading the building block on a solid support (This step is not necessary if the building block already comprises a solid support)

b) stepwise conjugating one or more amino acids to functionality B (this is done by standard solid phase synthesis methods, preferably Fmoc solid phase synthesis)

c) removing the protecting group of functionality C

d) attaching the label to the reactive group deprotected in step c)

e) optionally deprotecting the amino protecting group of the N-terminal amino acid and attaching a label to said amino group (This step is only performed if the peptide shall also comprise an N-terminal label)

f) optionally cleaving the C-terminally labelled peptide from the solid support.

The present invention further relates to a method for preparing C-terminally labelled peptides using a building block according to the present invention which already comprises one or more labels by

a) optionally loading the building block comprising one or more labels on a solid support (This step is not necessary if the building block already comprises a solid support)

b) stepwise conjugating one or more amino acids to functionality B (this is done by standard solid phase synthesis methods, preferably Fmoc solid phase synthesis)

c) optionally deprotecting the amino protecting group of the N-terminal amino acid and attaching a label to said amino group (This step is only performed if the peptide shall also comprise an N-terminal label)

d) optionally cleaving the C-terminally labelled peptide from the solid support.

### DETAILED DESCRIPTION OF THE INVENTION

FIG. 1 shows the synthesis of fluorescence-quench peptide substrates according to the state of the art.

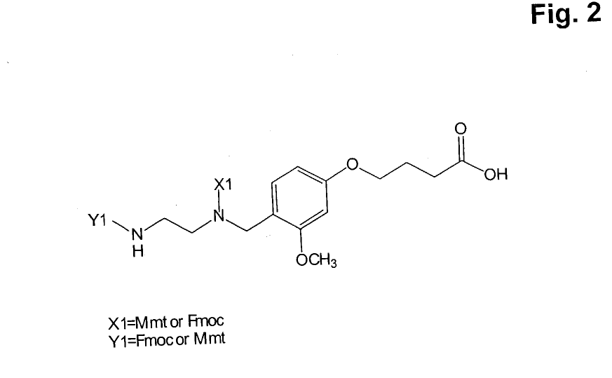

FIG. 2, 3 and 4 show preferred embodiments of the building block according to the present invention.

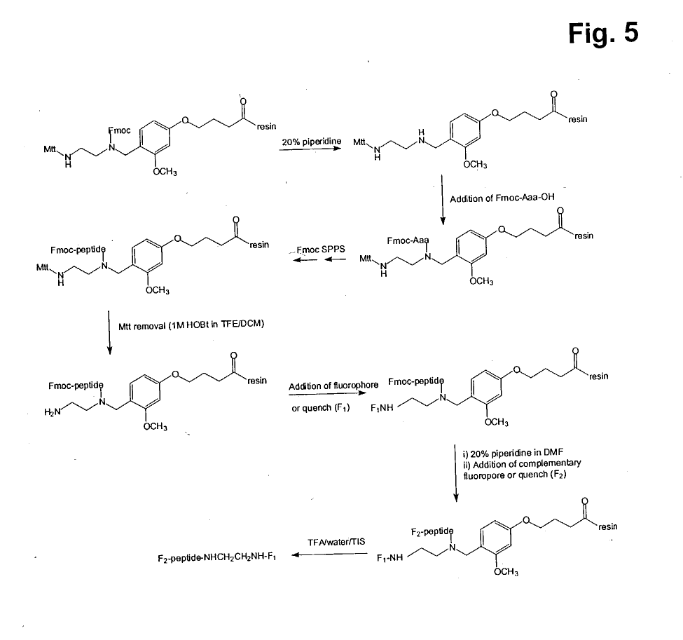

FIG. 5 gives an example of the inventive synthesis of a C-terminally labelled peptide.

A label is a chemical residue which is directly or indirectly able to generate a detectable signal, preferably a fluorescence, phosphorescence, luminescence, chemoluminescence or bioluminescence signal or a signal which can be detected visually or electrically. Further information about non-radioactive labels can e.g. be found in A. J. Garman “Non-radioactive Labelling” Academic Press, 1997. A label is also an affinity tag, such as biotin, or a chemically reactive group, such as NH2NH—, NH₂O—, —SH, —CHO, which could be used to selectively immobilise the tagged peptide to surfaces or beads carrying complementary reactive groups, or which can be further modified for labelling. Especially preferred labels are those cooperating as a fluorophore/quench pair.

A protecting group is attached to a chemical residue like an amino group, a hydroxyl group, a carboxyl group etc. to hinder this group from taking part in a chemical reaction. Protecting groups, suitable reaction conditions for introducing and cleaving these groups are known to a person skilled in the art. Further information about protecting groups which are commonly used in peptide synthesis can e.g. be found in “Fmoc Solid Phase Peptide Synthesis—A Practical Approach”, edited by W. C. Chang and P. D. White, Oxford University Press, 2000.

A solid support is any soluble or preferably insoluble material, e.g. a polymeric resin, a glass surface or bead or a plastic surface or bead suitable for peptide synthesis, e.g. polystyrene-, PEG/polystyrene- or cellulose resins, glass microscope slides or deep-well plates which are derivatized with e.g. amino-, hydroxy- or halo- groups. A survey about suitable resins can be found in Novabiochem® 2002/3 Catalog on solid phase peptide synthesis, synthesis notes, page 2.1-2.56.

Functionality means a residue, especially a chemical residue which has a given, defined function.

Heteroaryl means that one or more C-atoms of the arylic residue are substituted by N, O or S.

Concerning Linker K and/or L, Polyethylene Glycol Means (—O—CH₂—CH₂)_{x to y} (x=1, y=20).

A substituted alkyl chain is an alkyl chain comprising one or more C-atoms which are independently from one another preferably substituted by —C1 —C8-alkyl, —C5—C20-aryl, —C6—C30-arylalkyl, —C6—C30-alkylaryl, —OH, —O—C1—C8-alkyl, —O—C5—C20-aryl, —O—C6—C30-arylalkyl, —O—C6—C30-alkylaryl, —SH, —S—C1—C8-alkyl, —S—C5—C20-aryl, —S—C6-C30-arylalkyl, —S—C6—C30-alkylaryl, —NH2, —NH—C1—C8-alkyl, —NH—C5—C20-aryl, —NH—C6—C30-arylalkyl, —NH—C6—C30-alkylaryl, N(C1—C8-alkyl)₂, —COOH, CONH₂, —COO—C1—C8-alkyl, —COO—C5—C20-aryl, —COO—C6-C30-arylalkyl, —COO—C6-C30-alkylaryl, —F, —Cl, —Br, —I or —NO₂.

The building block according to the present invention provides an easy way for preparing C-terminally labelled peptides. It provides a central trivalent nitrogen atom to which is attached

a) a means for attachment to the solid support

b) a means for attachment of one or more labels

c) a means for attachment of one or more amino acids or peptides

A general formula of the building block is given in formula I.

In the following the main features of the building block according to the present invention are described in more detail:

a) Means for Attachment to the Solid Support (Functionality A in Formula 1)

Peptides are generally prepared by solid phase synthesis. The first amino acid is bound to the solid support via its carboxyl group. This carboxyl group is not set free until the peptide is split off from the solid support.

Consequently, for C-terminally labelling peptides during solid phase synthesis, the label has to be attached to a residue other than the terminal carboxyl group, or attached to the carboxyl group of the protected peptide in solution. As a consequence, C-terminally labelling peptides is often quite complicated and laborious. According to the present invention, this problem is solved by providing a building block which

connects the carboxyl group of the first amino acid with the solid support,

provides a means for attachment of a label

is stably connected to the C-terminus of the peptide even when the peptide is deprotected and split off from the solid support.

Functionality A of the building block according to the present invention is responsible for the connection to the solid support. It comprises the solid support or a chemical residue suitable for binding to a solid support. Suitable residues are e.g. residues which are reactive towards amines, alcohols or halides, like COOH, OH, SH, NCS, NCO, NH₂. Suitable residues as well as suitable reaction conditions for binding to a solid support are known to a person skilled in the art.

The second important feature of functionality A is a means for cleavage from the solid support. That means A typically comprises a cleavage site which, after completion of the attachment of amino acids and/or one or more labels to the building block, releases the rest of the building block (i.e. the trivalent nitrogen atom with functionality B and C, comprising one or more amino acids and one or more C-terminal labels) from the solid support. Suitable cleavage sites and cleavage conditions are known from solid phase peptide synthesis and are disclosed e.g. in Novabiochem® 2002/3 Catalog on solid phase peptide synthesis, synthesis notes, page 2.1-2.66.

In a preferred embodiment, the cleavage site is a highly electron-rich benzyl compound as shown in formula II. This benzyl compound is especially suitable to withstand conditions for attachment of amino acids or labels as well as for removal of protecting groups used in these syntheses, especially the reaction conditions used in Fmoc peptide synthesis. On the other hand, it is easily cleaved under acidic conditions.

In a very preferred embodiment, A is a residue according to formula II, with R¹=MeO, R²−R³=H, R⁴=MeO, Z=H and X=O, most preferably a residue with R¹=MeO, R²−R⁴=H, Z=H and X=O.

b) Means for Attachment of Amino Acids (Functionality B in Formula I)

Functionality B is the site for the attachment of the peptide chain. In solid phase peptide synthesis, typically, the peptide chain is build up by stepwise conjugating one amino acid after the other. Protocols for peptide synthesis are known to a person skilled in the art. Details about Fmoc solid phase peptide synthesis can e.g. be found in “Fmoc Solid Phase Peptide Synthesis—A Practical Approach”, edited by W. C. Chang and P. D. White, Oxford University Press, 2000.

Functionality B can per se comprise one or more amino acids.

Nevertheless, preferably, at the start, it does not comprise any amino acid. Amino acids to be attached to the building block according to the present invention, are conjugated to a reactive group of the building block via their carboxyl group. The reactive group of the building block can be the central trivalent nitrogen atom (in case there is no linker L) or a nitrogen atom, preferably an amino group, linked to the central trivalent nitrogen atom via a linker L. Consequently, if B does not comprise an amino acid, it preferably is an amino group or an amino protecting group or an amino group carrying a protecting group.

A schematic view of two preferred residues is given below:

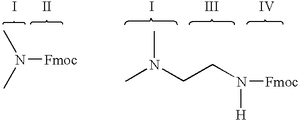

In the left scheme, the central trivalent nitrogen atom is marked with “I”, “II” marks functionality A which is in this case an amino protecting group. In the right scheme, the central trivalent nitrogen atom is again marked with “I”, “III” marks an alkyl linker L, “IV” marks a different implementation of functionality A—an Fmoc protected amino group.

Linker L may also be branched. A branched linker L provides the possibility to attach more than one peptide to the building block. In this case, the two and more functionalities B may be different or identical. In the schematic view below, a branched linker is shown with two sites for the attachment of a functionality B. In the left scheme, one functionality B is NH—P¹ with P¹ being e.g. a base-labile amino protecting group, the other functionality B is NH—P² With P² being an amino protecting group to be cleaved under conditions different from those needed for the cleavage of P¹. In the right scheme, both functionalities B are identical. As a consequence, this construction is suitable for parallel deprotection of the amino group and parallel conjugation of e.g. amino acids.

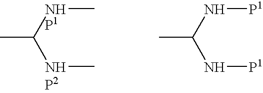

c) Means for Attachment of Label(s) (Functionality C in Formula I)

Functionality C is the site for attachment of one or more labels. C might be directly connected to the central trivalent nitrogen atom or it might be connected with the central trivalent nitrogen atom via a linker K. Functionality C can already comprise one or more labels. This is especially suitable if the label shall be introduced prior to synthesis of the peptide chain. In this case, a building block comprising one or more labels is attached to a solid support. After solid phase synthesis of the peptide chain, the product—a C-terminally labelled peptide—is split off from the solid support. If the building block does not already comprise one or more labels, A typically is a residue suitable for the attachment of a label, preferably a reactive group, a protected reactive group or, if there is no linker M, a protecting group which is directly connected with the central trivalent nitrogen atom. Preferred reactive groups are NH₂, OH, SH, hydrazino, hydroxylamine or aldehyde groups.

Linker K may be linear or branched. A branched linker K provides the possibility to attach more than one functionality C, i.e. more than one label, to the building block. In this case, the two and more functionalities C may be different or identical allowing the attachment of two and more different or identical labels.

d) Linker K and L

The building block according to the present invention comprises at least one linker K and/or L. By choosing a linker K and/or L of a certain type, the properties of the building block can be influenced. As an example, the length of the linker may influence the distance between the peptide chain and the label. The polarity of the linker may influence the solubility of the building block. Substituting an alkyl linker L by a polyethylene glycol linker, for example, would greatly increase the solubility of the peptide which is attached to the linker in polar solvents.

In addition, as discussed above, a branched linker offers the possibility of attaching more than one peptide chain and/or more than one label.

It is of course necessary to make sure that the functionalities A, B and C can be selectively reacted with other functionalities to be introduced into the building block (e.g. solid phase, labels or amino acids). For this reason, protecting groups have to be chosen that can be selectively cleaved without an unwanted influence on the rest of the molecule. Table 1 provides several examples of protecting groups or functionalities than can be combined. Suitable cleavage conditions for the protecting groups or cleavage sites shown in Table 1 are listed in brackets after the respective group:

TABLE 1
Fmoc can in all cases be also substituted
for other base-labile protecting groups such
as 2-(4-nitrophenylsulfonyl)ethoxycarbonyl (NSC) group.

| Protecting group | | |
|---|---|---|
| for chain | Protecting group for | Linker to solid |
| extension (*¹) | attachment of label (*²) | support (*³) |
| Mmt | Fmoc (piperidine) | 4-(4-Formyl-3- |
| (HOBt in | | methoxyphenoxy) |
| TFE/DCM or DCA | | alkyl (TFA) |
| in DCM) | | |
| Alloc (Pd(O)) | Fmoc (piperidine) | 4-(4-Formyl-3- |
| | | methoxyphenoxy) |
| | | alkyl (TFA) |
| ivDde, Dde | Fmoc (piperidine) | 4-(4-Formyl-3- |
| (hydrazine in DMF) | | methoxyphenoxy) |
| | | alkyl (TFA) |
| Mmt (HOBt in | Alloc (Pd(0)) | 4-(4-Formyl-3- |
| TFE/DCM or DCE | | methoxyphenoxy) |
| in DCM) | | alkyl (TFA) |
| Fmoc (piperidine) | Alloc (Pd(0)) | 4-(4-Formyl-3- |
| | | methoxyphenoxy) |
| | | alkyl (TFA) |
| Mtt, Bpoc (1% TFA | Fmoc (piperidine) | 4-(4-Formyl-3- |
| in DCM) | | methoxyphenoxy) |
| | | alkyl (TFA) |
| Mtt, Bpoc (1% TFA | Alloc (Pd(0)) | 4-(4-Formyl-3- |
| in DCM) | | methoxyphenoxy) |
| | | alkyl (TFA) |

*¹This protecting group may be identical with or comprised in functionality B. It is split off for the attachment of amino acids for peptide chain extension. It may also be the temporary protecting group of the amino group of the amino acids which are introduced for peptide chain extension.
*²This protecting group may be identical with or comprised in functionality C. It is split off for the attachment of a label.
*³this chemical residue is part of functionality A. It provides a suitable cleavage site for cleavage from the solid support.

FIG. 2, 3 and 4 show preferred embodiments of the building block according to the present invention.

FIG. 2 shows a preferred building block for universal usage. Functionality A comprises a terminal carboxyl group for attachment to a solid support. It further comprises the preferred 4-(4-Formyl-3-methoxyphenoxy)alkyl cleavage site.

Depending on the protecting groups X1 and Y1, the amino acids for peptide synthesis might be attached directly to the central trivalent nitrogen atom or to the aminoalkyl group. Preferably, the site of attachment of amino acids (functionality B) is the site comprising the Fmoc protecting group.

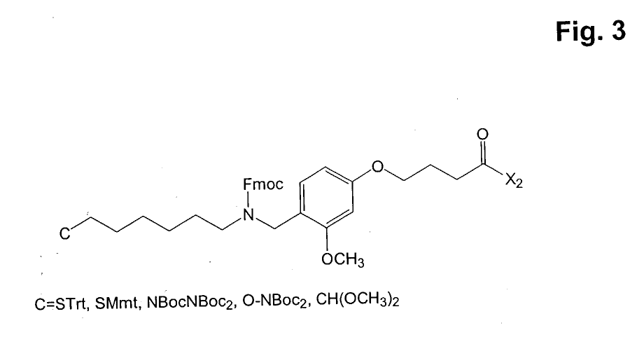

FIG. 3 shows another preferred building block already comprising a solid support. The attachment to the solid support (X2) is done via the preferred 4-(4-Formyl-3-methoxyphenoxy)alkyl cleavage site. Functionality B for the attachment of amino acids is an Fmoc protecting group being directly attached to the central trivalent nitrogen atom. Functionality C is connected to the central trivalent nitrogen atom via a C6-alkyl linker K. Functionality C is a protected SH, hydrazino, hydroxylamine or aldehyde group. A C-terminal thiol group can e.g. be used to attach peptides to gold-coated surfaces to produce tools for bioassay. Hydrazino or hydroxylamine groups can be selectively reacted with aldehyde functions. Conversely, peptides containing C-terminal aldehyde groups can be reacted with e.g. peptides, proteins, DNA or carbohydrate molecules, or surfaces derivatized with hydrazino or hydroxylamine functions.

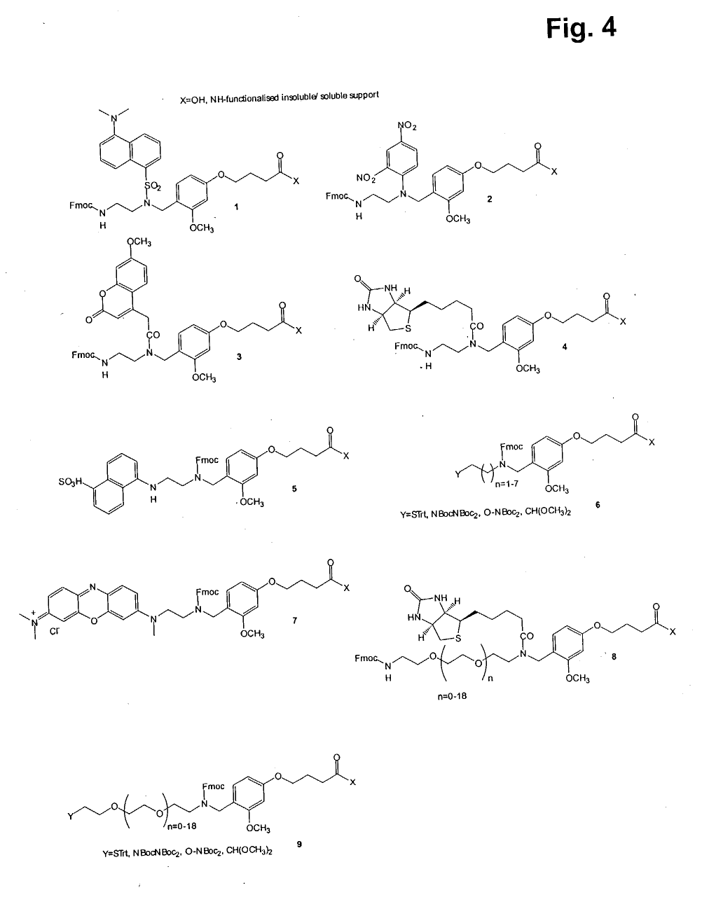

FIG. 4 shows nine preferred building blocks already comprising a label. X3 is OH so that the building block comprises a terminal carboxyl group for attachment to a solid support or the solid support. In case X3 is OH, the attachment to the solid support can be performed by reacting the terminal carboxyl group of functionality A with an OH- or NH-functionalized solid support.

After attachment to the solid support, the Fmoc protecting group of functionality B is split off and amino acids can be conjugated to the building block.

The invention further relates to a method for preparing C-terminally labelled peptides. The steps which are necessary for preparing the C-terminally labelled peptide depend on the building block which is used. Therefore, several examples are given using different building blocks.

1) Synthesis Using the Universal Building Block According to FIG. 2

This synthesis procedure is suitable when building blocks are used which do not contain a solid support, a label or amino acids, yet. In a first step, the building block is attached to a suitable OH- or NH-functionalized solid support. Afterwards, peptides can be prepared, using Fmoc solid phase synthesis methods. At the end of chain extension, the Mmt group is removed and any carboxylic acid, sulphonyl chloride, acid chloride, isocyanate, or isothiocyanate-containing label, e.g. a fluorophore or quench group, can be attached. The N-terminal Fmoc group is then removed and in case of synthesis of fluorescence-quench peptide synthesis, the complementary carboxylic acid, sulphonyl chloride, acid chloride, isocyanate, or isothiocyanate-containing fluorophore or quench group can be attached to the N-terminus of the peptide.

If a portion of the resin with Mmt and N-terminal Fmoc groups is retained, the process can be repeated using different fluorophore/quench pairs until the optimum configuration of fluorophore and quench is obtained for a particular application. Instead of fluorophores, biotin, or any other group containing a carboxylic acid, isocyanate, isothiocyanate, sulphonyl chloride and acid chloride may also to be attached. With biotin, this would allow from a single synthesis the effects on avidin binding of placing the biotin group at the N- or C terminus of a peptide to be evaluated, and how these different modes of presentation of the peptide effect the bioassay.

Finally, optionally, the labelled peptide is split off from the solid support. For some applications, e.g. if the solid support is a glass microscope slide or a deep-well plate to be used in some assay formats, the labelled peptide is left bound to the solid support.

An example for the application of the universal linker according to FIG. 2 is shown in FIG. 5. In FIG. 5, “resin” means solid support.

The method for preparing C-terminally labelled peptides using a building block which does not comprise a label therefore comprises the following general steps:

a) optionally loading the building block on a solid support (This step is not necessary if the building block already comprises a solid support)

b) conjugating the amino acids/peptide to functionality B (this is done by standard solid phase synthesis methods, preferably Fmoc solid phase synthesis)

c) removing the protecting group being comprised in functionality C

d) attaching the label to the reactive group deprotected in step c)

e) optionally deprotecting the amino protecting group of the N-terminal amino acid and attaching a label to said amino group (This step is only performed if the peptide shall also comprise an N-terminal label)

f) optionally cleaving the C-terminally labelled peptide from the solid support.

Step a) and b) may also comprise the cleavage of one or more protecting groups. A person skilled in the art of peptide synthesis knows when and how to perform such cleaving steps for loading on a solid support or peptide chain extension. A person skilled in art further knows which amino acid derivatives (amino acids carrying protecting groups) are used for solid phase peptide synthesis.

2) Synthesis Using a Pre-Formed Building Block Already Comprising a Solid Support (e.g. According to FIG. 4)

In many instances it may be advantageous to have a label, e.g. a chromogenic group or affinity tag, such as biotin, already attached to the building block. Examples for such building blocks are given in FIG. 4. The building block can then be attached directly to e.g. an amino functionalized solid support (e.g. resin, glass, plastic or cellulose surface), eliminating the chemical steps required to add the label. The building block can be configured such that, following removal of the Fmoc group, the site of attachment of peptide is a primary amine, as in structures 1-4 of FIG. 4. This arrangement avoids the difficult step of loading the first amino acid to the sterically hindered central trivalent nitrogen atom, as is the case with e.g. structure 6 in FIG. 4.

The pre-formed building block approach is particularly advantageous for structures containing biotin and EDANS, respectively (e.g. structures according to building block 4 and 5 of FIG. 4). Peptides derivatized with EDANS on the C-terminal carboxyl group cannot be prepared by using the universal linker according to FIG. 2, i.e. by introducing the label after peptide chain synthesis, as EDANS does not possess a carboxyl group. The EDANS fluorophore is usually added to the C-terminal carboxyl group of peptides by coupling, in solution, a protected peptide bearing a free C-terminal carboxyl group with EDANS. The linker 5 enables, therefore, such peptides to be prepared by standard solid phase methods which are much more rapid and amenable, especially to library synthesis.

The synthesis of biotinylated peptides by solid phase synthesis is difficult owing to the poor solubility of biotin and biotinylated derivatives, such as Fmoc-Lys(biotin)-OH and biotin-OSu. Building block 4 avoids these difficulties as the biotin is already in place and peptides are prepared by standard methods on the building block's primary amino group.

Building blocks already carrying one or more labels are particularly suited to preparing peptides in multiple array formats, such as on cellulose discs, as the building block comprising the label is added in a single step, avoiding multiple reactions at multiple sites.

The method for preparing C-terminally labelled peptides using a building block which already comprises one or more labels therefore comprises the following general steps:

a) optionally loading the building block comprising one or more labels on a solid support (This step is not necessary if the building block already comprises a solid support)

b) conjugating the amino acids/peptide to functionality B (this is done by standard solid phase synthesis methods, preferably Fmoc solid phase synthesis)

c) optionally deprotecting the amino protecting group of the N-terminal amino acid and attaching a label to said amino group (This step is only performed if the peptide shall also comprise an N-terminal label)

d) optionally cleaving the C-terminally labelled peptide from the solid support.

The building block and the method for preparing C-terminally labelled peptides according to the present invention therefore offer the following advantages over prior art:

1) Less synthetic steps are needed for the preparation of C-terminally labelled peptides as the inventive building blocks can be easily prepared and can e.g. already comprise the label.

2) The building blocks according to the present invention offer high synthetic flexibility as one can decide whether the label is favourably introduced prior or after peptide synthesis.

3) When introducing the label after peptide synthesis, one can separate the loaded solid support into several portions. Each portion can be modified with a different label thus generating multiple differently labelled peptides out of one peptide synthesis.

4) Due to the high flexibility of the building block, more than one peptide or more than one label can be attached.

5) The linkers K and/or L can be used to influence the properties of the C-terminally labelled peptide, e.g. by choosing linkers of specific length or polarity.

The building block and method according to the present invention can be used for preparing C-terminally labelled peptides by solid phase synthesis in single substrate, substrate array and library format.

Without further elaboration, it is believed that one skilled in the art can, using the preceding description, utilize the present invention to its fullest extent. The preferred specific embodiments and examples are, therefore, to be construed as merely illustrative, and not limitative to the remainder of the disclosure in any way whatsoever.

The entire disclosures of all applications, patents, and publications cited above and below are hereby incorporated by reference.

### EXAMPLES

I. Mmt-ethylenediamine-MPB—OH(C₃₄H₃₈N₂O₅; mw: 554.69)

Materials:

| | |
|---|---|
| 1) 34.7 g 4-(4-Formyl-3-methoxy-phenoxy)-butyric | 145.7 mM |
| acid (mw: 238.2) | |
| 2) 100 ml DMF | |
| 3) 9.19 g NaOMe (mw: 54.02) | 170.1 mM |
| 4) 300 ml Methanol | |
| 5) 48.4 g mono-Mmt-ethylenediamine (oil, mw: 332.45) | ˜145 mM |
| 6) 250 ml Trimethylorthoformate | |
| 7) 69.8 g Na[(AcO)₃BH] (mw: 211.94) | 329 mM |

1, 2, 3 and 4 are mixed in a vessel and stirred for 10 min., giving a clear solution. 5 and 6 are added and stirring is continued for further 20 min. (yellow solution). ⅔ of 7 are added slowly within 20 min., while the pH dropped to approximately 6.4. Another 10 g of 3 are added to rise the pH to 7.4 (cooling with ice-bath). After adding the remaining portion of 7, the pH is adjusted again with 140 ml aqueous Na₂CO₃-solution (10%) to 7.2. Stirring is continued overnight. After that the pH has to be adjusted again to 7.2-7.4 and the organic solvents are removed partially under reduced pressure to yield a slightly thick solution with some wax-like particles of impurity (bis-Mmt-ethylene-diamine from 5). After filtration the solution is added to 1 l of ice-water under mechanical stirring to give a white precipitate. The slurry is stirred for further 30 min., filtered off, washed with water and dried under high vacuum over CaCl₂.

Yield: 70.4 g (87.1%); purity (HPLC, pH 6.0): 90%

II/1 N-Dansyl-N′-Mmt-ethylenediamine-MPB—OH: (C₄₆H₄₉N₃O₇S; mw:787.98):

Materials:

| | |
|---|---|
| 1) 9.43 g Mmt-ethylenediamine-MPB-OH (mw: 554.69) | 17 mM |
| 2) 0.2 g Imidazole (mw: 68.08) | 3 mM |
| 3) 60 ml Acetonitrile p.a. (dried over molecular sieves 3 Å) | |
| 4) 4.4 ml BSA (mv: 244.5) | 18 mM |
| 5) 5.6 ml Triethylamine (mv: 139) | 40 mM |
| 6) 4.7 g Dansylchloride (mw: 269.75) | 17.4 mM |
| dissolved in 35 ml Dioxane | |

1, 2, 3 and 4 are mixed in a vessel under strictly anhydrous conditions (dry solvents, drying tube) at r.t. until a clear solution is obtained. 5 is added and the mixture is cooled to 0° C. 6 is now added to the reaction mixture over a period of 30 min. Upon completion, the ice-bath is removed and the whole mixture is stirred for 1 h at r.t. The reaction was found to be complete after this time (checked by TLC, chloroform:methanol 4:1). 30 ml of methanol are added and stirring is continued for an additional hour. The solvents are removed under reduced pressure and the oily residue is dissolved in 50 ml of water and 12 ml of Na₂CO₃-solution (10%) to give a clear solution. The aqueous solution is extracted one time with diisopropyl ether and twice with diethyl ether (30 ml each) (one reextraction with water) and added dropwise under vigorous stirring to an ice-cold solution of 20 g NaH₂PO₄ in 150 ml ice-water, where a yellow, crystalline precipitate is formed. After stirring for an additional 15 min., the crystals are filtered, washed with water and dried under high vacuum.

Yield: 11.75 g (87.7%); purity (HPLC, pH 6.0)-95%); MS (found): 788.4 (M+H)+

II/2 N-Dansyl-N′-Fmoc-ethylenediamine-MPB-AM-resin:

a) Loading:

Materials:

| | |
|---|---|
| 1) 15 g Aminomethyl-PS resin (01-64-0143; 0.9 mMole/g) | 13.5 mM |
| 2) 10.3 g N-Dansyl-N′-Mmt-ethylenediamine-MPB-OH | 13.1 mM |
| (mw: 787.98) | |
| 3) 250 ml DMF | |
| 4) 5.65 ml DIPEA (mv: 171.2) | 33.0 mM |
| 5) 7.8 g PyBOP (mw: 520.3) | 15.0 mM |
| 6) 1.5 g Acetylimidazole (mw: 110.12) | 13.6 mM |

1 and 2 are suspended in 3. 4 and 5 are added and the resulting slurry is agitated overnight at r.t. The rate of loading was determined by TNBS-test of the beads to be slightly positive. Testing of the mother liquor by TLC showed that almost no linker 2 was left uncoupled. (Under these conditions, the estimated loading of the final product would be aprox. 0.5 mMole/g). Then 6 is added together with 2 ml of 4 and agitation is continued for another 2 h (endcapping) until the TNBS-test is negative. The beads are filtered off and washed in the following sequence: 3×DMF, 3× water, 3×isopropanol, 3×THF, 2×isopropanol, 4×methanol (L-10), 2×ether, 3×hexane and dried under high vacuum at ambient temperature.

Yield: 26 g

b) Mmt-Fmoc-conversion:

To deprotect the Mmt-function, the resin is preswollen in 200 ml dry DCM for 20 min. at r.t., followed by the addition of a prepared solution of 10 g HOBt×H₂O in 30 ml DCM and 70 ml TFE. The slurry is agitated for 2 h at ambient temperature. After filtration, the resin is transferred back into the vessel and the cleaving procedure is repeated for 1 h with the same amount of fresh reagent-mix. Then the resin is filtered again and washed in the following way: 2×DCM, 3×isopropanol, 3×THF (TNBS-test: positive). After transferring the resin back into the vessel, 250 ml DMF are added with 10.5 g Fmoc-OSu (31 mM; mw: 337.3) and 3.3 ml N-methylmorpholine (30 mM; mv:110.2). The slurry is agitated overnight (a TNBS-test was negative), filtered, washed and dried as described in paragraph a).

Yield: 25.5 g; Fmoc-determination: 0.51 mMole/g

III/1 N-Mca-N′-Mmt-ethylenediamine-MPB—OH(C₄₆H₄₆NO₉; mw:770.88):

a) Materials:

| | |
|---|---|
| 1) 2.77 g Mmt-ethylenediamine-MPB-OH (mw: 554.69) | 5.0 mM |
| 2) 20 ml pyridine | |
| 3) 20 ml DMF | |
| 4) 1.85 g Mca-OSu (mw: 331.28) | 5.58 mM |

1 is dissolved in 2 and 3 at r.t. 4 is added and the mixture is stirred overnight until the reaction is complete by TLC (chloroform:methanol 4:1). Half of the solvent is removed under reduced pressure. The reaction mixture is diluted with 20 ml water and precipitated by adding this solution dropwise into an ice-cold solution of 8 g NaH₂PO₄ (32 mM; mw:156.01) in 100 ml water. To remove traces of starting material 1, the crude product is filtered off, dissolved in 150 ml EtOAc, dried over Na₂SO₄ and filtered over a short column of silica (˜25 g). Elution is completed by an additional 350 ml of EtOAc. After removal of the solvent, the crude product is dried to a foam and stirred with isopropyl ether to yield a crystalline material, which can be filtered and dried.

Yield: 2.00 g (52%); purity ˜90%.

b) Materials:

| | |
|---|---|
| 1) 5.55 g Mmt-ethylenediamine-MPB-OH (mw: 554.69) | 10 mM |
| 2) 50 ml dry DMF | |
| 3) 2.5 ml pyridine (mv: ˜81) | 30 mM |
| 4) 50 mg imidazole (mw: 68.08) (cat.) | 0.73 mM |
| 5) 2.7 ml BSA (mv: 244.5) | 11 mM |
| 6) 3.65 g Mca-OSu (mw: 331.28) | 11 mM |

1, 2, 3 and 4 are mixed in a vessel at r.t. under strictly anhydrous conditions. 5 is added and the mixture is stirred until a clear solution is formed. After addition of 6, the solution is allowed to stir overnight at r.t. until the reaction is found to be complete by TLC (9A). The solvent is removed under reduced pressure and the oily residue is dissolved in a mixture of 20 ml methanol, 15 ml Na₂CO₃-solution (10%) and 40 ml of water. This solution is extracted twice with ether (re-extraction with water) and added dropwise to solution of 26 g NaH₂PO₄ in 1100 ml ice/water under vigorous stirring. The crude, gluey product is extracted three times with EtOAc, separated from the aqueous layer, dried over Na₂SO₄ and isolated (after removal of the solvents) as an oil. Traces of starting material 1 are removed completely by filtration of a 5% solution of the oil in EtOAc over a short column with 15 g silica and subsequent washing with the same solvent. The purified material is isolated, after removal of the solvent, as a foam. Subsequent stirring of this foam with diisopropyl ether yields a nicely crystalline product.

Yield: 6.8 g (88%); purity ˜90%; MS (found): 771.3 (M+H)+

III/2 N-Mca-N′-Fmoc-ethylenediamine-MPB-AM-resin:

a)Loading:

Materials:

| | |
|---|---|
| 1) 12 g Aminomethyl-PS resin (01-64-0143; 0.9 mMole/g) | 10.8 mM |
| 2) 6.0 g N-Mca-N′-Mmt-ethylenediamine-MPB-OH | 7.79 mM |
| (mw: 770.88) | |
| 3) 130 ml DMF | |
| 4) 4.6 ml DIPEA (mv: 171.2) | 26.8 mM |
| 5) 4.5 g PyBOP (mw: 520.3) | 8.65 mM |

Resin-loading and end-capping is carried out by the same procedure as described above in II/1.

Yield: 18.12 g

b)Mmt-Fmoc-conversion:

The conversion of the protecting groups is carried out as described above in II/1.

Yield: 17.3 g; Fmoc-determination: 0.36 mMole/g

IV/1 N-Biotinyl-N′-Mmt-ethylenediamine-MPB—OH(C₄₄H₅₂N₄O₇S; mw 780.985):

Materials:

| | |
|---|---|
| 1) 7.6 g Mmt-ethylenediamine-MPB-OH (mw. 554.69) | 13.7 mM |
| 2) 60 ml DMF | |
| 3) 3.2 ml pyridine | |
| 4) 3.4 ml BSA (mw: 244.5) | 13.9 mM |
| 5) 4.78 g Biotinyl-OSu (01-63-0106; mw: 341.39) | 14.0 mM |

1, 2, and 3 are mixed and stirred in a vessel with magnetic stirrer under argon ensuring strictly anhydrous conditions at r.t. until a clear solution is formed. 4 is added and the stirring continued overnight. After this time the reaction is complete (test by TLC (chloroform/methanol 4:1)). The resulting solution is concentrated by rotary-evaporation, mixed with 20 ml of methanol, 20 ml Na₂CO₃-solution (10%) and 40 ml of water and extracted three times with ether (30 ml each, 1×reextraction with water). The combined aqueous phases are added dropwise to an ice-cold solution of 26 g NaH₂PO₄ in 150 ml water. The crude gum-like precipitate is redissolved in EtOAc (extraction of the aqueous layer), extracted with water, dried over sodium sulfate and silica, concentrated and crystallised by adding ether to the residue. After filtration, the material is dried under high vacuum at r.t.

Yield: 8.5 g (79%); purity (HPLC) ˜86%; MS (found): 781.4 (M+H)+

IV/2. N-Biotinyl-N′-Fmoc-ethylenediamine-MPB-AM-resin:

a) Loading

Materials:

| | |
|---|---|
| 1) 8.5 g N-Biotinyl-N′-Mmt-ethylenediamine-MPB-OH | ˜10 mM |
| (mw: 780.99) | |
| 2) 12 g Aminomethyl-PS resin (01-64-0143; 085 mMol/g) | 10.2 mM |
| 3) 5.3 ml DIPEA (mw: 171.2) | 31 mM |
| 4) 180 ml DMF | |
| 5) 6.2 g PyBOP (mw: 520.3) | 11.9 mM |

Resin-loading and end-capping is carried out under argon by the same procedure as described above in II/1.

Yield: 21.88 g.

b) Mmt-Fmoc-conversion:

The conversion of the protecting groups is carried out under argon as described above in II/1.

Yield: 20.93 g; Fmoc-loading.: 0.49 mMole/g; Loading by elemental sulphur analysis: S: 0.44 mMole/g.

V/1 N—DNP—N′—Mmt-ethylenediamine-MPB—ONa (C₄₀H₃₉N₄O₉Na; mw; 742.76):

Materials:

| | |
|---|---|
| 1) 10 g Mmt-ethylenediamine-MPB-OH (mw. 554.69) | 18 mM |
| 2) 0.2 g imidazole (mw: 68.08) | 3 mM |
| 3) 60 ml dry acetonitrile | |
| 4) 5 ml BSA (mw: 244.5) | 20.45 mM |
| 5) 6.9 ml DIPEA (mw: 171.2) | 40.3 mM |
| 6) 3.74 g 2,4-Dinitro-fluorobenzene (mw: 186.1) | 20 mM |

1, 2, 3 and 4 are mixed under strictly anhydrous conditions until a solution is formed. 5 is added and the mixture is cooled down to 0° C. 6 is dissolved in ˜20 ml dry acetonitrile and added dropwise to the stirred solution in about 20 min. Then the ice-bath is removed and the solution is allowed to warm up to r.t. within 2 h. The reaction is determined by TLC (chloroform:methanol 4:1) and found to be complete after this time. After adding 50 ml of methanol, the yellow solution is concentrated to an oil by rotary-evaporation and precipitated with isopropanol (100 ml) to form a gum-like residue at the bottom of the flask. The supernatant is poured away and the crude product is dissolved in 100 ml EtOAc and 10 ml acetonitrile. The organic layer is washed 3-times with NaH₂PO₄-solution (10% in water) and water, dried over Na₂SO₄, concentrated to an oil and re-dissolved in 100 ml of isopropanol/acetonitrile (4:1). By adding 1.1 g NaOMe in 20 ml methanol, the corresponding Na-salt is formed as a gluey residue. After removal of the supernatant, the residue is rinsed with isopropanol until it becomes brittle, stirred to form a powdered material, filtered off and dried under high vacuum at r.t.

Yield: 11.57 g (˜86%); purity (HPLC): ˜85%; MS (found): 721.3 (w) (M+H)⁺; 743 (vw) (M+Na)⁺(A single nonpolar impurity of ˜15% was found to be unaffected by the following coupling step).

V/2 N—DNP—N′—Fmoc-ethylenediamine-MPB-AM-resin:

a)Loading:

Materials:

| | |
|---|---|
| 1) 15 g Aminomethyl-PS resin | 12.75 mM |
| (01-64-0143; 0.85 mMole/g) | |
| 2) 11.57 g N-DNP-N′-Mmt-ethylenediamine-MPB-ONa | ˜13 mM |
| (mw: 742.76) | |
| 3) 200 ml DMF | |
| 4) 7 ml DIPEA (mw: 171.2) | 40.8 mM |
| 5) 8.0 g PyBOP (mw: 520.3) | 15.4 mM |

Resin-loading and end-capping is carried out by the same procedure as described above in II/1.

Yield: 25.4 g

c) Mmt-Fmoc-conversion:

The conversion of the protecting groups is carried out as described above in II/1. Due to the enhanced acid-sensitivity of this product, the cleaving-time is reduced to 2×30 min. by using only 9 g (1^{st} cleavage) and 4.5 g (2^{nd} cleavage) HOBt·H₂O.

Yield: 23.9 g; Fmoc-determination: 0.40 mMole/g

VI/1 EDANS-MPB—OH(C₂₄H₂₈N₂O₇S; mw: 488.56);

Materials

| | |
|---|---|
| 1) 7.99 g 1,5-EDANS* (mw: 266.3) | 30 mM |
| 2) 100 ml methanol and 10 ml water | |
| 3) 14 ml Benzyl-trimethylammonium-hydroxide | 33 mM |
| (40% in H₂O; mv˜418) | |
| 4) 16.5 ml DIPEA (91-0058; mv: 171.2) | 95 mM |
| 5) 8.14 g 4-(4-Formyl-3-methoxy-phenoxy)-butyric acid | 34 mM |
| (01-60-0061; mw: 238.2) | |
| 6) 16 g Na[(AcO)₃BH] (90-0521; mw: 211.94) | 75 mM |

*5-(2-Aminoethylamino)-1-naphtalenesulfonic acid

1 is dissolved in a mixture of 2, 3 and 4 until a clear solution is formed; 5 is added at once and the mixture is allowed to stir 20 min. at r.t. After cooling down to ˜4° C. (ice bath), 6 is added carefully in about 20 min. By stirring overnight, the reaction-mixture is allowed to warm up at ambient temperature. Checking by TLC (chloroform:methanol:32% acetic acid 15:4:1), the reaction is found to be complete (pH: ˜6.4). By adding 40 ml of hydrochloric acid (6 N), the pH is adjusted to ˜4.0, where the product is crystallising as an inner salt. The slurry is stirred 1 h at r.t. and 1 h in an ice bath, filtered off, washed with ice-water, acetonitrile and ether and dried under high vacuum at r.t.

Yield: 11.8 g (80%)

VI/2 Fmoc-EDANS-MPB—OH (C₃₉H₃₈N₂O₉S; mw:710.802):

Materials

| | |
|---|---|
| 1) 12.6 g EDANS-MPB-OH (mw: 488.56) | 25.79 mM |
| 2) 8.78 g Fmoc-OSu (mw: 337.3) | 26.03 mM |
| 3) 100 ml THF | |
| 4) 4.37 g NaHCO₃ (mw: 84.02) in 50 ml H₂O | 52.0 mM |

1 and 2 are suspended in 3 at r.t. 4 is added drop-wise to the stirred mixture in about 15 min. Stirring is continued overnight, resulting in a clear solution. The reaction is found to be complete by TLC (chloroform:methanol: 32% aqueous acetic acid 15:4:1) after this time. The volume is reduced to ⅓ by rotary-evaporation, diluted with 100 ml of water and extracted three-times with ether (30 ml each; 1×re-extraction with 20 ml water). After adding 15 g of NaH₂PO₄ (mw: 156.01; ˜100 mM), the pH of the aqueous layer is adjusted to 3.5-4, at which the product crystallises (after filtration, a second fraction is earned from the mother-liquor). The crude material is dried and recrystallised from DMF/acetonitrile.

Yield: 18 g (25.3 mM): 98%; purity ˜90%; MS (found): 711.3 (M+H)+

VI/3 EDANS-MPB-AM-resin:

a)Loading:

Materials:

| | |
|---|---|
| 1) 16.6 g Fmoc-EDANS-MPB-OH (mw: 710.802) | 23.5 mM |
| 2) 200 ml DMF | |
| 3) 3.95 ml DIPEA (mv: 171.2) | 22.9 mM |
| 4) 4.58 g HOBt aq. (mw. 153.15) | 30 mM |
| 5) 22.8 g Aminomethyl-PS resin | 19.4 mM |
| (01-64-0143; 0.85 mMole/g) | |
| 6) 13.5 g PyBOP (mw: 520.3) | 25.9 mM |
| 7) 15 ml DIPEA (mv: 171.2) | 87 mM |

1 is mixed with 2 and 3 until a clear solution is formed. 4 and 5 are added and the resulting slurry is shaken for 5 min. After addition of 6 and 7, the mixture is allowed to agitate for 3 h. The reaction is followed by TNBS-test and found to be nearly complete. End-capping is carried out by adding 10 g of Boc₂O (mw: 218.23; 45.8 mM). After 20 min. of agitating, the reaction is found to be complete (TNBS-test negative). Washing of the resin is carried out by the usual method described above.

Yield: 44.18 g; Fmoc-determination: 0.50 mMole/g.

b) Fmoc-deprotection:

Materials:

| | |
|---|---|
| 1) Fmoc-EDANS-MPB-AM-resin (0.5 mMole/g) | 23 mM |
| 2) 450 ml DMF | |
| 3) 10 ml DBU (mv: 149.5) | 66.9 mM |

1 is mixed with 2 and the resulting slurry was allowed to stand for 1 h at r.t. 3 is added and the mixture is agitated for further 20 min. After filtration and washing (3×DMF), the resin is transferred back to the vessel and the same cleaving-procedure is repeated one more time. Final washing and drying of the resin is carried out by our standard method, described above for II/1.

Yield: 41 g; loading (calculated): 0.54 mMole/g; loading by elemental sulphur analysis: S: 0.55 mMole/g; strongly positive chloroanil-test: deep-green beads (the corresponding Fmoc-protected resin gave negative result; no-colour change).

A test-loading of the deprotected resin with Fmoc—Leu—OH, PyBrOP and DIPEA (3 equivalents each, reaction time 3 h at r.t.) is in accordance with the theoretical data.

VII Synthesis of H—Asp—Glu—Val—Asp—Ala—Arg—NHCH₂CH₂— (biotinyl)-NH

H—Asp(OtBu)—Glu(OtBu)—Val—Asp(OtBu)—Ala—Arg(Pmc)—N HCH₂CH₂—(biotinyl)—N—MPB—AM resin was prepared on an ABi automated peptide synthesiser using N-Biotinyl-N′-Fmoc-ethylenediamine-MPB-AM-resin using standard Fastmoc protocols. The peptide was cleaved from the resin by treatment with TFA/TIPS/water (95:2.5:2.5) for 3 hours. The TFA was evaporated and the product precipitated with ether. The HPLC profile of the crude biotinylated peptide is shown in FIG. 6. ES-MS analysis confirmed the major peak at time 10.51 min. to be the desired product (expected M+1 972; found 972).

[image]

FIG. 6: HPLC Profile of crude H—Asp—Glu—Val—Asp—Ala—Arg—NHCH₂CH₂—(biotinyl)—NH. Column: Vydac C18 peptide protein column. Gradient: 5-100% in 30 min. Buffers: A, 0.06% TFA in water; B, 90:10:0.06 MeCN:water:TFA. Detection: 220 nm.

VIII Synthesis of H—Lys(Dnp)—Asp—Glu—Val—Asp—Ala—Arg—EDANS

H—Lys(Dnp)—Asp(OtBu)—Glu(OtBu)—Val—Asp(OtBu)—Ala—Arg(Pmc)—EDANS—MPB—AM resin was prepared on an ABi automated peptide synthesiser using EDANS—MPB—AM—resin using standard Fastmoc protocols. The peptide was cleaved from the resin by treatment with TFA/TIPS/water (95:2.5:2.5) for 3 hours. The TFA was evaporated and the product precipitated with ether. The HPLC profile of the crude biotinylated peptide is shown in FIG. 7. ES-MS analysis confirmed the major peak at time 10.04 min. to be the desired product (expected M+1 1203; found 1203).

[image]

FIG. 7: HPLC Profile of H—Lys(Dnp)—Asp—Glu—Val—Asp—Ala—Arg—EDANS. Column: Vydac C18 peptide protein column. Gradient: 5-100% in 30 min. Buffers: A, 0.06% TFA in water; B, 90:10:0.06 MeCN:water:TFA. Detection: 220 nm.

### Abbreviations

Alloc allyloxycarbonyl

AM-resin aminomethylated polystyrene

Bpoc 2-(4-Biphenyl)-isopropyloxycarbonyl

BSA bis-N,O-trimethylsilylacetamide

Dabsyl 4-(4-Dimethylaminophenylazo)benzoic acid

Dansyl 5-dimethylaminonaphthalenesulfonyl

DCA dichloroacetic acid

DCM dichloromethane

DIPEA diisopropylethylamine

DMF dimethylormamide

Dnp 2,4-dinitrophenyl

EDANS 2-(5-sulfonaphthal-1-yl-amino)ethylamine

ES electrospray

EtOAc ethyl acetate

Fmoc 9-fluorenylmethoxycarbonyl

HOBt N-Hydroxybenzotriazole

HPLC high pressure liquid chromatography

ivDde 1-(4,4-dimethyl-2,6-dioxocyclohex-1-ylidene)-3-methylbutyl

Mca (7-methoxycoumarin-4-yl)acetyl

Mmt 4-methoxytrityl

MPB [4-(3-carboxypropyloxy)-2-methoxy-phenyl]methyl

MS mass spectrum

Mtt 4-methyltrityl

OSu N-oxysuccinimide

Pmc 2,2,5,7,8-pentamethylchroman-6-sulfonyl

PyBOP benzotriazole-1-yl-oxy-tris-pyrrolidino-phosphonium hexafluorophosphate

PyBrOP bromo-tris-pyrrolidino-phosphonium hexafluorophosphate

r.t. room temperature

TFA trifluoroacetic acid

TFE trifluoroethanol

THF tetrahydrofuran

TIPS triisopropylsilane

TLC thin layer chromatography

TNBS 2,4,6-trinitrobenzenesulfonic acid

We claim:

1. Building block for preparing C-terminally labelled peptides by solid phase peptide synthesis according to formula I 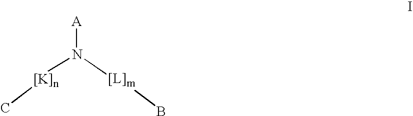
wherein
A is a functionality for the attachment to a solid support or a functionality already comprising a solid support
B is a functionality for the attachment of one or more amino acid or peptides or a functionality already comprising one or more amino acids or peptides
C is a functionality for the attachment of one or more labels or a functionality already comprising one or more labels,
K and L are independently from one another a linear or branched, substituted or unsubstituted alkyl chain with at least two C-atoms, whereby one or more non-neighbouring C-atoms might be substituted by O, NH, N—(C1-C6)Alkyl, N—(C5-C15)Aryl, S, a carbonyl group, ester group or an amide group and/or neighbouring C-atoms might be connected via a double or triple bond:
m, n are 0 or 1, whereby m+n is at least 1.

2. Building block according to claim 1, wherein B is an amino protecting group or a protected amino group

3. Building block according to claim 1, wherein C comprises one or more labels.

4. Building block according to claim 1, wherein m+n is 1.

5. Building block according to claim 1, wherein K and L are independently from one another C₂—C₈-alkyl or —(O—CH₂—CH₂—)_{q}— with q=1 to 20.

6. Building block according to claim 1, wherein A is a residue according to formula II 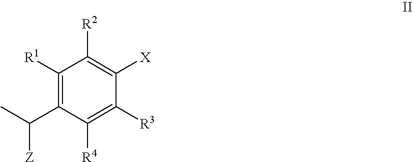
whereby
R¹, R², R³ and R⁴ independently from one another are H, C₁—C₈ alkyl, C₁—C₈ alkoxy, C₅—C₁₈ aryl or heteroaryl or C₅—C₁₈ aryloxy or heteroaryloxy,
X is a functionality for attachment to the solid support or a functionality already comprising a solid support.
Z is H, C₁—C₈-alkyl, C₅—C₂₀ aryl or C₅—C₂₀ heteroaryl.

7. Building block according to claim 6, wherein X is a residue according to formula III
—D—R⁵—E III
with
D being CH₂, S, NH or O
R⁵ being C—C₁₀ alkyl
E being COOH, OH, SH, NCS, NCO, NH₂, halide (Cl, Br, I) or the solid support.

8. Method for preparing C-terminally labelled peptides using a building block according to claim 1 by
a) optionally loading the building block on a solid support
b) stepwise conjugating one or more amino acids to functionality B
c) removing the protecting group of functionality C
d) attaching the label to the reactive group deprotected in step c)
e) optionally deprotecting the amino protecting group of the N-terminal amino acid and attaching a label to said amino group
f) optionally cleaving the C-terminally labelled peptide from the solid support.

9. Method for preparing C-terminally labelled, peptides using a building block according to claim 3 by
a) optionally loading the building block comprising one or more labels on a solid support
b) stepwise conjugating one or more amino acids to functionality B
c) optionally deprotecting the amino protecting group of the N-terminal amino acid and attaching a label to said amino group
d) optionally cleaving the C-terminally labelled peptide from the solid support.
